(19) United States
(12) Patent Application Publication
(10) Pub. No.: US 2014/0107223 A1
(43) Pub. Date: Apr. 17, 2014

(54) TOPICAL UBIQUINOL SUPPLEMENT SKIN CARE COMPOSITIONS

(71) Applicant: WHITEHILL ORAL TECHNOLOGIES, INC., Stafford, TX (US)

(72) Inventor: Dale G. BROWN, Wharton, TX (US)

(73) Assignee: WhiteHill Oral Technologies, Inc., Stafford, TX (US)

(21) Appl. No.: 13/651,050

(22) Filed: Oct. 12, 2012

(51) Int. Cl. A61K 31/09; A61P 17/00; A61Q 19/00; A61K 8/891

(52) U.S. Cl. 514/720

(57) ABSTRACT

Topical, UBIQUINOL supplement compositions useful for treating skin by: reducing oxidative stress, adjunctively restoring “local” UBIQUINOL levels, revitalizing aged and/or U.V. damaged skin and regulating immune responses, including responses to inflammatory challenges; comprising: stabilized UBIQUINOL supplement, an emulsion of polydimethylsiloxane in a nonionic surfactant and skin penetrants.

### FIELD OF THE INVENTION

The present invention is directed to stabilized, UBIQUINOL supplement, skin care compositions suitable for reducing oxidative stress, adjunctively restoring “local” UBIQUINOL levels in the skin, revitalizing skin indicating aging and/or exposure to U.V., and responding to inflammatory challenges.

### BACKGROUND OF THE INVENTION

UBIQUINOL is the reduced, active antioxidant form of coenzyme Q10 (CoQ10). Produced naturally within healthy bodies, UBIQUINOL is CoQ10 that has been converted (activated by the addition of two electrons).

UBIQUINOL is considered to be the strongest lipid soluble antioxidant that is biosynthesized, to provide an active defense against oxidative insult to lipids, protein and DNA; while maintaining redox balance. See: THE POWER OF UBIQUINOL, by Dr. Robert Barry, Ph.D. (2010), Health Point Press, Sherman Oaks, Calif. 91303.

UBIQUINOL supplement, such as Kaneka QH™ UBIQUINOL SUPPLEMENT is unstable in the presence of oxygen and light. This instability has limited UBIQUINOL SUPPLEMENT use since its commercial introduction in 2008 to oral administration via gelatin capsules. R&D efforts from 2008 to the present by many companies, research organizations, etc., attempting to stabilize UBIQUINOL supplement for topical administration to the skin have been unsuccessful.

### OBJECTS OF THE INVENTION

An object of the present invention is to stabilize UBIQUINOL supplement in the presence of oxygen and light.

Another object of the invention is to develop a manufacturing process suitable for producing light- and oxygen-stable UBIQUINOL supplements useful for topical administration.

Yet another object of the invention is to develop topical UBIQUINOL supplement compositions suitable for dispensing as crèmes, balms, lotions, ointments, etc., onto skin for adjunctively supplementing and restoring “local” UBIQUINOL levels.

Another object of the invention is to: reduce oxidative stress, adjunctively restore UBIQUINOL levels, respond to inflammatory challenges, revitalize aged and/or U.V. damaged skin.

### SUMMARY OF THE INVENTION

The present invention is directed to stable, topical, UBIQUINOL supplement, skin care compositions useful for reducing oxidative stress and adjunctively restoring “local” UBIQUINOL levels.

The UBIQUINOL in stable, UBIQUINOL supplement, skin care compositions of the present invention is represented by the following structural formula:

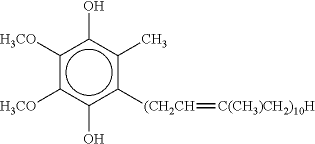

Stable UBIQUINOL supplements for skin care compositions of the present invention include a stabilizing composition comprising: ascorbyl palmitate in a weight ratio to UBIQUINOL from between about 0.75 and 2.5 and an emulsion of polydimethylsiloxane in a nonionic surfactant.

The stable, UBIQUINOL supplement, skin care composition also includes trans-dermal absorption facilitators compositions.

The present invention is directed to a stable, topical, UBIQUINOL, skin supplement compositions useful in: reducing oxidative stress, restoring “local” UBIQUINOL levels, revitalizing aging and/or U.V. damaged skin and responding to inflammatory challenges, comprising:
an emulsion of polydimethylsiloxane in a surfactant;
an effective level of UBIQUINOL supplement;
stabilizing compositions for UBIQUINOL supplement; and
trans-dermal, absorption facilitator compositions, wherein:
   upon application to the skin, said stable, UBIQUINOL composition penetrates the skin:
   (a) reducing oxidative stress;
   (b) restoring and maintaining adequate levels of UBIQUINOL;
   (c) revitalizing aging skin;
   (d) responding to inflammatory challenges; and
   (e) repairing U.V. damaged skin.

Specifically, stable UBIQUINOL supplement compositions of the invention, upon topical administration to the skin, effect passive diffusion through the skin, reducing oxidative stress by restoring UBIQUINOL levels and revitalizing aging skin.

### The Role of Stable, UBIQUINOL Supplement, Skin Care Compositions is to: Adjunctively Restore UBIQUINOL Levels, Revitalize Skin and Respond to Inflammatory Challenges

CoQ10 is a fat soluble, essential, quinone molecule, found in every cell, tissue and organ in the body. CoQ10 partners with other enzymes in the body and plays a vital role in cellular and bodily health, including: energy production and free radical production. CoQ10 production in the body decreases with aging. CoQ10 has been shown to have antioxidant potential and to promote ATP production in the mitochondria inner membrane.

Oxidized (ubiquinone) and reduced (ubiquinol) forms have been identified for CoQ10. Ubiquinone is converted by NADPH-dependent CoQ10 reduction, which uses NADPH as an electron donor, into UBIQUINOL. UBIQUINOL is known to exist as the active form of the coenzyme in the body. In a study in which ubiquinone was orally administered to rats, most CoQ10 molecules detected from the lymph were in the form of UBIQUINOL, suggesting that the coenzyme is reduced immediately after being absorbed from the intestinal tract. UBIQUINOL molecules circulating in the body are incorporated into lipoproteins in the liver and are distributed to tissues all over the body via the blood stream.

These molecules appear to be converted to oxidized molecules in the blood when exposed to oxidative stress caused by various factors. However, since the ubiquinone molecules are re-reduced in the liver, over 90% of all CoQ10 molecules present in the blood of a healthy person are in the form of UBIQUINOL, suggesting that the molecules are in a strong reduction condition.

It is well established that CoQ10 (ubiquinone) is not well absorbed into the body, as has been published in many peer-reviewed, scientific journals. Since the reduced CoQ10 (ubiquinol) form has two additional hydrogens, it results in the conversion of two ketone groups into hydroxyl groups on the action portion of the molecule. This causes an increase in the polarity of the CoQ10 molecule and may be a significant factor behind the observed enhanced bioavailability of UBIQUINOL. Orally, UBIQUINOL exhibits greater bioavailability than ubiquinone: 150 mg per day of UBIQUINOL in a softgel resulted in peak blood values of 3.84 mcg/ml within 28 days. Reduced CoQ10 is absorbed faster and in a larger amount than oxidized CoQ10. See U.S. Pat. No. 6,184,255 assigned to KANEKA CORP.

Oxidative stress is detectable as changes in plasma CoQ10 concentrations and composition and plays an important role in aging of skin, inflammation, etc. For example, deficiencies of Coenzyme Q10 (CoQ10), both oxidized (ubiquinone) and reduced (ubiquinol), have been implicated in: skin aging, U.V. damage of skin and various skin inflammations. “Local” oxidative stress of the skin is associated with wrinkled skin.

UBIQUINOL is the first lipid soluble antioxidant available for antioxidant defenses of the skin associated with oxidative stress. Stable, UBIQUINOL supplements applied topically to the skin via compositions of the invention, would be the first lipid soluble, antioxidant response to oxidative stress in the skin. In this regard, the plasma redox status of UBIQUINOL in the “local” systemic circulation of the oral cavity provides a measure of “local” systemic oxidative stress.

Adjunctive UBIQUINOL mediated effects on “local” skin inflammatory markers, with the topical, stable, UBIQUINOL supplement, skin care compositions of the invention, are expected to indicate reductions in the secretion of several pro-inflammatory cytokines. Damage to nuclear or mitochondrial DNA, indicated by mitochondrial dysfunction caused by oxidative stress is proposed as a common link among various inflammatory and oxidative stress conditions of the skin.

The role of UBIQUINOL supplement responding to inflammatory challenges is particularly well documented in the adjunctive UBIQUINOL supplement treatment of inflammatory challenges to the oral mucosa. The literature references cited below apply with equal force to responding to oxidative stress and inflammatory challenges associated with the skin, where reducing oxidative stress and restoration of UBIQUINOL levels is achieved via topical administration of stable, UBIQUINOL supplement, skin care products of the invention.

Gingivitis and periodontitis are inflammatory disorders caused by bacteria living in biofilm. It is known that oxidative stress in the bloodstream and gingiva is increased by oral inflammatory disorders, including: gingivitis and periodontitis. The net effect of this oxidative stress . . . UBIQUINOL deficiencies, which are to be relieved with the compositions of the invention. See:
Littaru, et. al. “Deficiency of coenzyme Q10 in gingival tissue from patients with periodontal disease.” (1971) Proc. Nat. Acad. Science USA 68:2332-2335.
Nakamura, et. al. “Deficiency of coenzyme Q10 in gingiva of patients with periodontal disease.” (1973) Int. J. Vit. Nut. Res. 43:84-92.

Therapy of gum disease with UBIQUINOL and coenzyme Q10 (CoQ10) is reported by:
Folkers K (1992) “A critique of 25 years of research which culminated in the successful therapy of periodontal disease with coenzyme Q10.” J. Dent. Health 42:258-263.
McCree, et. al. (1993) “Therapy with coenzyme Q10 for patients with periodontal disease.
Effect of coenzyme Q10 on subgingival microorganisms.” J. Dent. Health 43:659-666.
Hanioka, et. al. “Effect of Topical Application of Coenzyme Q10 on Adult Periodontitis.” (1994 Molec. Aspects of Med. Vo. 85 (Supplement) pp. S241-S248.
KANEKA Corp./Nihon University collaborative research on “Effect of the reduced form of coenzyme Q10 (ubiquinol) on oral environment for periodontal disease” presented by K. Sugawara and N. Sugano to: “The 63^{rd} Meeting of the Vitamin Society in Japan” held Jun. 4 and 5, 2011 in Hiroshima.

It is well established that oral inflammations, including gingivitis an periodontitis, are accompanied by a deficiency of coenzyme Q10 (both oxidized and reduced versions).

Hanioka, et. al. (1994) topically applied CoQ10 once weekly via syringe to periodontal sites, for six weeks. The authors reported:
“Tremendous improvement was found in bleeding on probing at CoQ10 treated sites after topical application was provided in combination with mechanical debridement.”
“Sites bled at day 0 showed no bleeding at six weeks. The effect of treatment was statistically significant only at experimental sites after mechanical subgingival debridement.”
“When topical application was provided as a sole treatment, periodontal probing depth, clinical attachment level and gingival crevicular fluid flow showed improvement only at CoQ10 treated sites.”
“Thus topical application of CoQ10 might enhance resistance of periodontal tissue to periodontopathic microorganisms.”
“improves adult periodontitis not only as a sole treatment but also in combination with traditional nonsurgical periodontal therapy.”
However, the amount of CoQ10 absorbed in gingival tissue was not determined in this pilot study.

A June 2011, KANEKA/Nihon University presentation reports that oral administration of UBIQUINOL @ 150 mg capsule/day for two months “is effective in improving oral environment for periodontal disease.”

Folkers K. (1992) states:
“CoQ10 is, therefore, recommended for both prophylactic and therapeutic treatment of periodontal disease.”
“The indispensability of the intrinsic CoQ10 in bioenergetics was emphasized as the basis for the extraordinary healing and the dental benefits resulting from the administration of CoQ10 to periodontal patients.”
“It was concluded that CoQ10 can improve bioenergetics and can be prophylactically and adjunctively used for extraordinary healing during routine periodontal therapy.”
“It was concluded that this CoQ10 therapy to reduce periodontal disease and particularly microorganisms is preferable to ordinary treatment with antibacterial agents because CoQ10 therapy improves the immune mechanisms to control disease.”

From 1994 to date, extensive, published research by Kaneka Corp. on CoQ10 has established:
(a) an increased absorption rate into the bloodstream for reduced coenzyme Q10 (ubiquinol) compared to oxidized CoQ10 (ubiquinone);
(b) there are stability issues affecting UBIQUINOL availability when it is exposed to air and/or light;
(c) procedures have been developed for accurately monitoring plasma levels of ubiquinone and UBIQUINOL; and
(d) a shift in the proportions of subgingival microorganisms of periodontitis patients is attributed to UBIQUINOL adjunctive therapy. Note: This is a key finding for proposed relief of oxidative stress based on administering adjunctive UBIQUINOL aqueous-free emulsion compositions onto the oral mucosa, as described and claimed in the present invention.

Additional relevant references include:
Nylander M. and Nordlund M. (1991). Clinical effects on periodontal status after given oral supplement of ubiquinone. Swed. J. Biol. Med. 1, 6-11.
Wilkinson E. G., Arnold R. M., Folkers K., Hansen I. and Kishi H. (1975). Bioenergetics in clinical medicine. II. Adjunctive treatment with coenzyme Q10 in periodontal therapy. Res. Com. Chem. Path. Pharm. 12, 111-124.
Wilkinson E. G., Arnold R. M. and Folkers K. (1976). Bioenergetics in clinical medicine. VI. Adjunctive treatment of periodontal disease with coenzyme Q10. Res. Com. Chem. Path. Pharm. 13, 715-719.
Hanioka T., Tanaka M., Ojima M., Shizukulski S. and Folkers K. (1994). Effect of topical application of coenzyme Q10 on adult periodontitis. Molec. Aspects Med. Vol. 15 (Supplement) S241-S248.
Kishi T., et. al. (1993). Journal of Dental Health. 43:667-672.
Shimura Y., et. al. (1981). Rinsho-to-Kenkyu, 58, 1349-1352.
U.S. Pat. Nos. 7,897,169; 7,303,921; and 6,184,255.

### Oxidative Stress in General and Adjunctive UBIQUINOL Supplement Skin Care Products

The present invention is directed to relieving “oxidative stress” and restoring UBIQUINOL levels associated with aging and/or U.V. damaged skin, inflammatory challenges of the skin, etc. Adjunctive, UBIQUINOL, skin care supplements, topically applied by the compositions of the present invention to the skin: reduce oxidative stress, restore UBIQUINOL levels and respond to inflammatory challenges, as evidenced by reduced circulating markers of inflammation. See: X. Wang, et. al. Am. J. Clinical. Nutr. 2004, September; 80(3):649-655:
“Co supplementation with Vitamin E and coenzyme Q10 reduces circulating markers of inflammation in baboons.” Vitamin E may be added to the compositions of the present invention.
“Inflammation and oxidative stress are processes that mark early metabolic abnormalities in vascular diseases.
Dietary supplementation with Vitamin E reduces baseline inflammatory status indicated by the CRP concentrations in healthy baboons. Cosupplementation with CoQ10 significantly enhances this anti-inflammatory effect of Vitamin E.”

Subsequent “inflammation” studies carried out with ubiquinol by C. Schmelzer, et. al. J. Clin. Biochem. Nutr. 44:62-66, January 2009, indicated:
“In vitro effects of the reduced form of coenzyme Q10 on secretion levels of TNF-α and chemokines in response to LPS in the human monocytic cellline THP-1”
“In conclusion, our results indicate anti-inflammatory effects of the reduced form of CoQ10 on various inflammatory cytokines and chemokines in vitro.”
“Ubiquinol, the reduced form of coenzyme Q10 serves as a potent antioxidant of lipid membranes.”

Topical administration of stable, UBIQUINOL supplement, skin care compositions of the invention is designed to maximize the therapeutic potential of stabilized UBIQUINOL supplement by adjunctively restoring “local” UBIQUINOL deficiencies within circulating lipoproteins at systemic “uptake” rates. Multiple, topical doses of skin care crèmes, balms, ointments, etc., throughout the day; provide an ongoing adjunctive response to “local” UBIQUINOL deficiencies caused by oxidative stress. These multiple, topical doses are projected to be responsive to systemic UBIQUINOL uptake. Such a controlled, adjunctive, dosing response to local UBIQUINOL deficiencies caused by oxidative stress is not available from orally administered UBIQUINOL supplement using one or more capsules of UBIQUINOL SUPPLEMENT daily.

Orally administered, UBIQUINOL supplement's low water solubility (less than 0.1 mg/ml) and high molecular weight of 865, results in:
(1) slow absorption of UBIQUINOL supplement from the gastrointestinal tract, i.e. approximately 6 hours required to reach peak concentration, with
(2) steady-state concentrations reached within two weeks of oral administration.

In contrast, topical, multiple dose administration of stable, UBIQUINOL supplement, skin care compositions of the invention relies on ongoing trans-dermal absorption to directly enter “local” systemic circulation (lymph system, bloodstream, gingiva, etc.). This alternative administration of stable, UBIQUINOL supplement, skin care compositions of the invention avoids the “first-pass drug effect,” which is experienced by orally administered drugs, where the drugs undergo metabolism. This “first pass drug effect” reduces the bioavailability of orally administered, stable UBIQUINOL supplement before it reaches systemic circulation. A therapeutic UBIQUINOL plasma level objective of >3.5 μg/ml is projected to be sufficient to respond to oxidative stress of the skin. The level of systemic oxidative stress associated with the skin can be established via the plasma redox status of UBIQUINOL.

Proposed advantages of multiple topical administrations of stable, UBIQUINOL supplement, skin care compositions of the invention from a crème, applied in repetitive doses throughout the day to oxidatively stressed skin; versus a single oral administration of UBIQUINOL supplement via capsule, include:
Efficiency of absorption into the skin increases as dose level decreases.
Bioavailability is optimized by avoiding “first-pass drug effect.”
Maximum therapeutic potential of stable UBIQUINOL supplement is achieved at a faster rate over a longer period of time.
“Local” UBIQUINOL systemic deficiencies are targeted directly vs. targeting UBIQUINOL deficiencies throughout the body.
Adjustments in topical administration can be made to accommodate varying UBIQUINOL plasma thresholds for different skin conditions.
Topical administration of stable UBIQUINOL supplement to the skin targets restoring “local” UBIQUINOL deficiencies via trans-dermal absorption vs. oral administration of stable UBIQUINOL supplement, which undergoes trans-mucosal absorption in the small intestine and targets restoring UBIQUINOL deficiencies throughout the body.
A single, topical, “local” administration of 10 to 20 mg of stable UBIQUINOL supplement compositions of the invention from a crème is absorbed rapidly with no indication of CoQ10 present. Multiple dosages throughout the day are responsive to stable UBIQUINOL supplement uptake in the systemic circulation and to the ongoing challenges posed by oxidative stress. This is in contrast to the single oral administration of a 100 to 200 mg capsule of stable UBIQUINOL supplement.
Multiple, topical administrations of stable, UBIQUINOL, skin care supplement, locally, totaling between 50 and 200 mg carried out over an 8 to 12 hour period; is a more effective response to oxidative stress of the skin. This extended topical administration is designed to optimize bioavailability while being responsive to local UBIQUINOL deficiencies attributed to continuing oxidative stress.

UBIQUINOL is considered to be the strongest lipid-soluble antioxidant that is biosynthesized, providing an active defense against oxidative insult to lipids, proteins and DNA.

UBIQUINOL supplement is unstable in the presence of oxygen, which has limited its use since its introduction in 2008 to oral capsules. R&D efforts, from 2008 to the present, by many companies attempting to stabilize UBIQUINOL for topical administration to skin have been unsuccessful.

The present invention represents a major R&D and manufacturing breakthrough in the stabilization and dispensing of Kaneka QH™ UBIQUINOL supplement for Topical applications to the skin, for relief of oxidative stress associated with: reduced UBIQUINOL levels, aging and/or U.V. damaged skin, inflammatory challenges, etc.

The present invention relies on transporting stabilized Kaneka QH™ UBIQUINOL supplement to the skin for diffusion into the “local” circulatory system. Proprietary: formulating, processing and dispensing conditions for this combination: assures that the oxidative properties of Kaneka QH™ UBIQUINOL supplement have not been compromised and that “reduced” Kaneka QH™ UBIQUINOL supplement is delivered topically to the skin.

Up to the present, restoration of UBIQUINOL deficiencies in the skin associated with oxidative stress has been primarily through adjunctive Kaneka QH™ UBIQUINOL supplement capsules administered orally.

The stabilized, Kaneka QH™ UBIQUINOL, skin care supplement, in combination with its dermal absorption facilitator, is rapidly diffused into the skin. The stabilized Kaneka QH™ UBIQUINOL supplement, combined with ULTRAMULSION® and an absorption facilitator, enters the “local” circulatory system via penetration through the skin. This topical, adjunctive administration of stable UBIQUINOL supplement is projected to help: restore “local” UBIQUINOL deficiencies, reduce oxidative stress and provide relief from inflammatory challenge, as discussed in the cited references.

This trans-dermal, stable absorption of Kaneka QH™ UBIQUINOL supplement, in the reduced state, continues until the topical crème is totally worked into the skin.

### Topical UBIQUINOL Supplement, Skin Care Compositions of the Invention Feature

Stabilized, Kaneka QH™, UBIQUINOL SUPPLEMENT, in combination with a proprietary, emulsion (ULTRAMULSION®). The stabilized, UBIQUINOL supplement remains in a reduced state as the stabilized UBIQUINOL supplement penetrates into the skin.
Topical, direct and rapid, adjunctive supplementation of “local”, depleted, UBIQUINOL levels in the skin result in the restoration of a healthy redox balance.
Stabilized, Kaneka QH™, UBIQUINOL supplement, in an emulsion of polydimethylsiloxane in nonionic surfactant, combined with a skin penetrant, ensures rapid, optimal absorption and assimilation by “local” skin.
Stable, UBIQUINOL supplement neutralizes free radicals in the “local” skin, reducing cellular damage of the skin that would otherwise contribute to or exacerbate aging of the skin.

### DETAILED DESCRIPTION OF THE PRESENTLY PREFERRED EMBODIMENTS

In a preferred embodiment of the invention, stable, UBIQUINOL supplement compositions are included in a topical skin care treatment of the invention, where UBIQUINOL supplement adjunctively administered: reduces oxidative stress by increasing “local” UBIQUINOL levels, and restores aging and U.V. damaged skin.

The present invention includes methods:
for treating inflammatory skin conditions, reducing oxidative stress, restoring UBIQUINOL levels, comprising topically administering stable, UBIQUINOL supplement compositions to the skin, comprising:
effective levels of UBIQUINOL supplement;
a stabilizing composition for UBIQUINOL comprising ascorbyl palmitate in a weight-ratio to UBIQUINOL from between about 0.75 and 2.5;
an emulsion of polydimethylsiloxane in a nonionic surfactant; and
trans-dermal penetrant facilitator compositions; where:
   upon application to the skin, said composition penetrates the skin, reducing oxidative stress and restoring “local” UBIQUINOL levels and responding to inflammatory challenges;
wherein application means for said topical, stabilized, UBIQUINOL, skin care supplement composition is selected from the group consisting of: crèmes, balms, ointments, lotions, etc.

For purposes of the present invention, suitable emulsions include ULTRAMULSIONS® that are comprised of polydimethylsiloxane in a nonionic surfactant, as described in the following U.S. Pat. Nos. 5,032,387; 5,098,711; 5,538,667 and 5,651,959; all of which are hereby incorporated by reference.

Preferred nonionic surfactants suitable for the invention are selected from the group consisting of: poloxamer 237, poloxamer 338, poloxamer 407 and combinations thereof.

For the purposes of the present invention, trans-dermal absorption facilitators are selected from the group consisting of: dexpanthenol, d-Limonene, poloxamer, PEG, benzyl alcohol, carbopol, chitosan, N-trimethylchitosan, menthol and combinations thereof.

Preferred emulsions for use as carriers of for stabilized UBIQUINOL supplement in the compositions of the present invention include emulsions of polydimethylsiloxane (PDMS) at viscosities ranging from between about 1000 cs and about 2.5 million cs. Particularly preferred, aqueous-free emulsions include as the discontinuous phase PDMS at viscosities between 10,500 cs and 2.5 million cs with those nonionic surfactants described in detail in U.S. Pat. No. 5,651,959, as the continuous phase.

Preferred emulsions for purposes of the present invention include those emulsions disclosed in U.S. Pat. Nos. 5,009,881; 5,032,387; 5,057,306; 5,057,307; 5,057,309; 5,538,667 and 5,651,959; all of which are included herein by reference.

Preferred polydimethylsiloxanes are selected from the group consisting of polydimethylsiloxane: at 1500 cs, at 12,500 cs, at 100,000 cs, at 250,000 cs, at 500,000 cs, at 750,000 cs, at 1.5 million cs, at 2.2 million cs, at 2.5 million cs and combinations thereof.

Preferred application means for the UBIQUINOL skin care supplement compositions of the present invention include: crèmes, ointments, balms, lotions, etc.

### Examples 1 Through 4

The present invention is further described by additional enclosed samples of stable UBIQUINOL supplement, skin care compositions of the invention.

### Example 1

### UBIQUINOL Skin Lotion

Tank A: 2 L stainless steel vessel was fitted with an overhead stirrer and an aluminum foil cover with constant purging with nitrogen. Deaerated water, 868.8 gm, was added and heated to 70 degrees C. with moderate stirring. The additional ingredients for this vessel were added: glycerin, 24 gm; sodium chloride, 12 gm; and oat kernel flour, 24 gm, were added and heated to 70 degrees C.

A 250 mL stainless steel beaker (Tank B) with overhead stirring and a nitrogen flush was heated to 70 degrees C. The following ingredients were added: cetyl alcohol, 30 gm; benzyl alcohol, 7.3 gm; methyl paraben, 2.4 mg; propyl paraben, 0.6 gm; isopropyl palmitate, 24 gm; petrolatum, 36 gm; and distearyldimonium chloride, 45 gm. After 10 minutes, the homogeneous contents were added to a 2 L stainless steel vessel (Tank C) fitted with overhead stifling and a nitrogen flush with heating to 70 degrees C.

A 100 mL stainless steel beaker (Tank D) was fitted with a nitrogen purge. An aqueous-free emulsion [poloxamer 338/polydimethylsiloxane (1000 cs)] (80:20), 84 gm, was added and heated to 70 degrees C. with overhead stifling. Propylene glycol, 24 gm, UBIQUINOL, 9 gm, and ascorbyl palmitate, 9 gm, were added to the beaker under a nitrogen blanket with stifling. When homogeneous, the contents of Tank D were added to Tank C under nitrogen with stirring and cooling to 30 degrees C. The white colored UBIQUINOL skin lotion was packaged under nitrogen in laminate tubes. Application of UBIQUINOL skin lotion to the forearm gave a silky smooth, moisturizing effect.

EXAMPLES 2 through 4 were prepared following the procedure detailed in Example 1:

| | Example 2 | Example 3 | Example 4 |
|---|---|---|---|
| | Product Type: | | |
| Ingredients | Crème | Balm | Ointment |
| water-dearated | qs | qs | Qs |
| glycerin | 4 | 2 | 2 |
| benzyl alcohol | 0.6 | 0.6 | 0.6 |
| dimethicone | 1.3 | 1.3 | 1 |
| poloxamer 338 | 8 | 5 | 6 |
| distearyldimonium chloride | 3 | 4 | 5 |
| micronized oat kernel flour | 1 | 1.5 | 2 |
| cetyl alcohol | 2 | 3 | 3.5 |
| isopropyl palmitate | 2.5 | 2.5 | 3 |
| petrolatum | 3 | 3 | 3 |
| sodium chloride | 1 | 1 | 1 |
| Ubiquinol | 0.5 | 0.6 | 0.75 |
| ascorbyl palmitate | 0.5 | 0.5 | 0.75 |
| propylene glycol | 2 | 2 | 2 |
| propyl paraben | 0.05 | 0.05 | 0.05 |
| methyl paraben | 0.2 | 0.2 | 0.2 |
| dexpanthenol | 1 | 3 | 5 |
| Spilanthes extract | 0.2 | 1 | 3 |

What is claimed is:

1. A stable Ubiquinol skin care composition, comprising:
(i) a polydimethylsiloxane-surfactant emulsion, wherein said polydimethylsiloxane is the discontinuous phase of said emulsion;
(ii) an effective level of Ubiquinol;
(iii) a stabilizing composition for the Ubiquinol; and
(iv) a trans-dermal absorption facilitator; wherein:
upon application to the skin, said composition penetrates the skin:
(a) reducing oxidative stress;
(b) restoring and maintaining adequate local levels of Ubiquinol;
(c) revitalizing skin;
(d) responding to inflammatory challenges; and
(e) restoring U.V. damaged skin; and
wherein, the Ubiquinol is represented by the structural formula

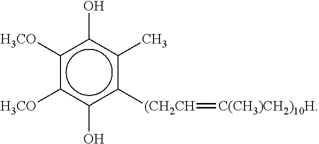

2. The composition according to claim 1, wherein said emulsion is aqueous-free and is comprised of polydimethylsiloxane emulsified in a nonionic surfactant.

3. (canceled)

4. The composition according to claim 1, wherein said stabilizing composition for Ubiquinol comprises ascorbyl palmitate and an emulsion of polydimethylsiloxane in a nonionic surfactant.

5. The composition according to claim 1, wherein said trans-dermal absorption facilitator composition is selected from the group consisting of dexpanthenol, d-Limonene, poloxamer, PEG, benzyl alcohol, carbopol, chitosan, N-trimethylchitosan, menthol and combinations thereof.

6. The composition according to claim 1, wherein application means for said skin care composition is selected from the group consisting of crèmes, lotions, ointments, balms and combinations thereof.

7. The composition according to claim 2, wherein said polydimethylsiloxane is selected from the group consisting of polydimethylsiloxane at 1000 cs, at 12,500 cs, at 100,000 cs, at 250,000 cs, at 500,000 cs, at 750,000 cs, at 1.5 million cs, at 2.2 million cs, at 2.5 million cs and combinations thereof.

8. The composition according to claim 2, wherein said nonionic surfactant is selected from the group consisting of poloxamer 237, poloxamer 338, poloxamer 407 and combinations thereof.

9. (canceled)

10. A method for treating oxidative stress; restoring Ubiquinol levels, revitalizing aged and/or U.V.-damaged skin; and responding to inflammatory challenges; comprising adjunctively administering a stabilized, Ubiquinol skin care composition onto a subject's skin, wherein the composition comprises:
(i) a polydimethylsiloxane-surfactant emulsion, wherein said polydimethylsiloxane is the discontinuous phase of said emulsion;
(ii) an effective level of Ubiquinol;
(iii) a stabilizing composition for the Ubiquinol; and
(iv) a trans-dermal absorption facilitator; wherein:
upon application to the skin, said composition penetrates the skin:
(a) reducing oxidative stress;
(b) restoring and maintaining adequate local levels of Ubiquinol;
(c) revitalizing skin;
(d) responding to inflammatory challenges; and
(e) restoring U.V. damaged skin; and
wherein, the Ubiquinol is represented by the structural formula

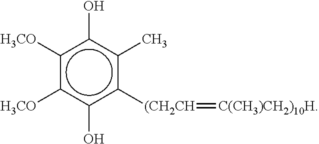

11. (canceled)

12. The method according to claim 10, wherein said trans-dermal penetration facilitator composition is selected from the group consisting of dexpanthenol, d-Limonene, poloxamer, PEG, benzyl alcohol, carbopol, chitosan, N-trimethylchitosan, menthol and combinations thereof.

13. The method according to claim 10, wherein application means for said topical skin care composition is selected from the group consisting of crèmes, ointments, lotions, balms and combinations thereof.

14. The method according to claim 10, wherein said composition is applied to the skin repeatedly throughout the day.

15. The composition according to claim 4, wherein the composition has a weight ratio of carboxymethylcellulose to Ubiquinol of between about 0.75 and 2.5.
